(19) United States
(12) Patent Application Publication
(10) Pub. No.: US 2006/0128666 A1
(43) Pub. Date: Jun. 15, 2006

(54) Use of sphingomyelin and lyso-sphingomyelin as absorption enhancers

(76) Inventor: Willem Ferdinand Nieuwenhuizen, Bunnik (NL)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/542,709

(22) PCT Filed: Jan. 20, 2004
(86) PCT No.: PCT/NL04/00047

§ 371 Date: Jul. 20, 2005

(30) Foreign Application Priority Data
Jan. 20, 2003 (NL) 1022442

(51) Int. Cl. A61K 31/685; A61K 31/663

(52) U.S. Cl. 514/78; 514/102

(57) ABSTRACT

The invention relates to agents to improve the uptake of nutrients and medicines, and more in particular to sphingomyelin and/or lysosphingomyelin for use in a pharmaceutical preparation to improve the uptake of certain nutrients and pharmaceutically active substances. The present invention provides a pharmaceutical preparation in which sphingomyelin and/or lysosphingomyelin is incorporated, a food provided with sphingomyelin and/or lysosphingomyelin and methods for the preparation thereof.

The invention relates to agents to improve the uptake of nutrients and medicines, and more in particular to sphingomyelin and/or lysosphingomyelin for use in a food composition or in a pharmaceutical preparation to improve the uptake of certain nutrients and pharmaceutically active substances. The present invention provides a pharmaceutical preparation in which a sphingomyelin and/or lysosphingomyelin is incorporated, a food provided with a sphingomyelin and/or lysosphingomyelin and methods for the preparation thereof

The uptake of food constituents and medicaments is accurately regulated by the body (stomach and intestinal epithelium). However, in some cases, the uptake control can be too strict. In particular, the uptake of some medicaments is (almost) completely blocked in this manner. In order to still achieve sufficient uptake, often, much higher doses of the medicaments are administered. Also, in many cases, chemical additives are used to improve the uptake of the medicines which are difficult to take up. These chemical additives may have undesired side effects.

An example of medicines which are difficult to take up are the bisphosphonates used in the control of osteoporosis. Of these medicines, less than 1% of the dose administered is actually absorbed in the blood. Bisphosphonates are harmful to the gastrointestinal tract and many complications of the use thereof are known.

It is desired to improve the uptake of medicines to thus limit the dose thereof to be administered and to reduce the possibility of the occurrence of side effects.

It has now surprisingly been found that certain natural lipids such as lysosphingomyelin can considerably improve the uptake of inter alia bisphosphonates.

It is known that certain sphingolipids, particularly sphingosine and ceramide, can influence the signal transduction cascade of body cells and thus affect the development and the metabolism of cells of the gastrointestinal tract.

Further, it is known that sphingomyelin reduces the absorption of cholesterol from the gastrointestinal tract.

It is all the more surprising that it has now been found that sphingomyelin and lysosphingomyelin can improve the uptake of substances from the gastrointestinal tract.

The present invention provides inter alia compositions and uses by which the uptake of the pharmaceutically active constituents from the gastrointestinal tract can be increased and by which the dose of the pharmaceutically active constituent to be administered can be limited.

Therefore, in a first aspect, the present invention relates to a pharmaceutical preparation to improve the uptake of a nutrient or pharmaceutically active substance comprising sphingomyelin and/or lysosphingomyelin or a precursor, a derivative or a pharmaceutically suitable salt thereof and one or more excipients.

Herein, a “derivative”, “analogon” or “analog” is defined as a sphingomyelin and/or lysosphingomyelin which has been subjected to chemical modification. Derivatization can comprise the substitution of certain chemical groups on, sphingomyelin and/or lysosphingomyelin. Such derivatizations are known from the state of the art. The derivatives and analogs maintain the biological activity of the natural lipid and function in a similar manner, but can offer advantages to the molecule such as a longer life, a resistance to decomposition or an increased activity.

Herein, a “pharmaceutically suitable salt” is defined as a salt in which the desired biological activity of the lipid is maintained and has minimal undesired toxicological effects. Non-limiting examples of such a salt are (a) acid addition salts formed with inorganic acids (for instance hydrochloric acid, hydrogen bromide, sulfuric acid, phosphoric acid, nitric acid, and the like), and salts formed with organic acids such as for instance acetic acid, oxalic acid, tartaric acid, succinic acid, malic acid, ascorbic acid, benzoic acid, tannic acid, palmitic acid, polyglutamic acid, naphthalene sulfonic acid, naphthalene disulfonic acid and polygalacturonic acid; (b) base addition salts formed with metal cations such as zinc, calcium, bismuth, barium, magnesium, aluminum, copper, cobalt, nickel, cadmium, sodium, potassium and the like, or with a cation formed from ammonia, N,N-dibenzylethylenediamine, D-glucosamine, tetraethylammonium or ethylenediamine; or (c) combinations of (a) and (b); for instance a zinc. tannate or the like.

The use of a pharmaceutically suitable salt of a sphingomyelin and/or lysosphingomyelin, such as an ammonium salt or a chloride salt, is preferred because the salt form strongly influences the solubility and thus the rapid availability of the compound. Preferably, a salt of HCl is used.

Herein, a “precursor” is defined as a derivative of which, specifically, the resistance against decomposition by, for instance, the digestive tract or other decomposition systems has been increased as a result of, for instance, chemical modification of the molecule.

It is possible to use the sphingomyelin and/or lysosphingomyelin in modified form, for instance by means of single or multiple methylation, acylation or acetylation or by modification to a formic acid amid.

Further, all possible racemates and (dia)stereoisomers of a sphingomyelin and/or lysosphingomyelin can be used in the present invention.

The chain length of the alkyl chain on the sphingomyelin base may vary. For lysosphingomyelin, a usual chain length is C₁₈, but substantially shorter or longer chains may also be used in embodiments of the present invention. The alkyl chains may be saturated, unsaturated, polyunsaturated or modified C₈-C₂₄ chains, optionally substituted with one or more groups chosen from the set consisting of hydroxyl, amino, alkylamino, arylamino, alkoxy, aryloxy, nitro, cyano, sulfonic acid, sulfate, sulfonate, phosphonate or phosphate, both unprotected and protected insofar as desired, and is known to a skilled person, for instance as described in Greene et al., Protective Groups in Organic Synthesis, John Wiley & Sons, 2^{nd} Edition, 1991. Suitable modifications comprise etherifications.

Preferably, lysosphingomyelin is used.

In addition to sphingomyelin and lysosphingomyelin themselves, which compounds comprise a choline head group to the sphingosylphosphoryl unit, derivatives of sphingomyelin and lysosphingomyelin may also be used in aspects according to the present invention. For instance, instead of a CH₂CH₂N⁺(CH₃)₃ choline group, also, for instance, other amino acids, preferably amino acids having a positive charge, may very suitably be used. Still more preferably, the choline group may be replaced by serine, ethanolamine or inositol, which compounds are, in that case, used as a head group. Most preferably, however, choline is used a head group.

In addition, analogs of sphingomyelin and lysosphingomyelin in which the choline has been replaced by ethanolamine, serine or inositol can be used in embodiments of the present invention.

It is further possible to use a combination of sphingomyelin and lysosphingomyelin in methods, compositions and uses according to the invention.

Essentially, sphingomyelin and lysosphingomyelin of any origin are suitable for use in embodiments according to the invention. Lysosphingomyelin and sphingomyelin may, for instance, be obtained from eggs. Further, the substances may be chemically manufactured or sphingomyelin may be isolated from, for instance, milk, soybeans, yeasts (essentially all species), bacteria, algae, plants, meat, brains, etc. for use in a pharmaceutical composition or food according to the invention. For use in a food according to the present invention, sphingomyelin and/or lysosphingomyelin is preferably obtained from so-called “food-grade” sources. Examples of such “food-grade” sources are inter alia baking yeast, brewer's yeast (in particular from draff) and egg, and certain types of bacteria, fungi, sponges and algae, particularly those species which are not toxic and preferably, but not exclusively, bacteria and fungi having a GRAS status (“Generally Recognized As Safe”).

Sphingomyelin and/or lysosphingomyelin can be obtained from above-described sources in manners known to a skilled person. In order to manufacture enriched fractions from these sources, the following can be used: extraction with (organic) solvents, chromatographic separation, precipitation, crystallization, and/or enzymatic or chemical hydrolysis. The production of a sphingomyelin-enriched fraction from milk is, for instance, known from WO94/18289. Sphingomyelin may also be obtained from fat concentrates of different animal products such as milk, egg and blood as known from U.S. Pat. No. 5,677,472. The lyso form can be chemically or enzymatically prepared from sphingomyelin. Methods for preparing sphingolipids and sphingolipid derivatives in general are known from inter alia EP 0 940 409, WO 98/03529 and WO 99/50433, and a skilled person will be able to manufacture derivatives in a known manner and to test these for increased activity, for more selective activity or for reduced side effects in order to obtain sphingolipid derivatives which can be used in the present invention.

A sphingomyelin and/or lysosphingomyelin, or a precursor, a derivative or a pharmaceutically suitable salt thereof may also be synthesized by known methods such as they are known from, for instance, U.S. Pat. Nos. 5,232,837 and 5,110,987, or by standard modifications of these methods.

A known problem associated with the administration of lipids is that they can be metabolized. This is particularly a problem for the use of lipids in the digestive tract. This problem can be solved by administering a sphingomyelin and/or lysosphingomyelin, or a derivative or a pharmaceutically suitable salt thereof, alone or in combination, as a so-called precursor compound comprising certain substituents so that this compound cannot be metabolized or can be metabolized to a lesser extent. The precursors are preferably resistant to hydrolysis in the upper parts of the digestive tract, and are, for instance, cleaved relatively easily in the cecum and/or colon, if the sphingomyelin and/or lysosphingomyelin needs to be active mainly in the cecum and/or colon. This increases the residual amount of compound at the location where the sphingomyelin and/or lysosphingomyelin is active. For instance, a sphingomyelin and/or lysosphingomyelin precursor can be used which can be cleaved or activated in vivo by a suitable enzyme so that a sphingomyelin and/or lysosphingomyelin is released which can promote the uptake of certain medicines. Such a method is inter alia known from WO 99/41266.

It is possible to modify a precursor of a sphingomyelin and/or lysosphingomyelin (or have it modified) by in situ enzymatic or chemical conversion, i.e. in the body, to a sphingomyelin and/or lysosphingomyelin which can be used in embodiments of the present invention. Such precursors of sphingomyeln and/or lysosphingomyelin are therefore also suitable for use according to the invention, on condition that the precursor is converted, in the body, preferably in the intestine, to sphingomyelin and/or lysosphingomyelin, for instance by enzymatic conversion, in which context, in situ activation is thus involved. It is, for instance, possible to administer the enzyme sphingomyelin deacylase together with, for instance, sphingomyelin, so that the sphingomyelin is converted to lysosphingomyelin, which is most preferably used in embodiments according to the present invention. Incidentally, the enzyme sphingomyelin deacylase is found in humans. Other examples of enzymes may, for instance, be found in Sueyoshi et al., 1997, J Lipid Res 38:1923-7. However, preferably, sphingomyelin and/or lysosphingomyelin are not used as precursors but in “active” form incorporated in a pharmaceutical preparation, a food or a nutritional supplement.

So, when, in the present description, the term “sphingomyelin and/or lysosphingomyelin” is used, this also refers to an analog or a derivative or a pharmaceutically suitable salt thereof, alone or in combination, or as a so-called precursor compound, unless explicitly stated otherwise.

Preferably, a pharmaceutical composition according to the invention is intended for or directed to oral administration. Compositions for oral administration will usually comprise an inert diluent or an edible carrier.

The compositions may be packaged in, for instance, gelatin capsules or may be tabletted in the form of tablets. For oral therapeutic administration, the active compound may be administered with excipients and be used in the form of, for instance, tablets, pastilles or capsules. Pharmaceutically suitable binding agents and/or adjuvants may also be added as constituents of the pharmaceutical composition.

The tablets, pills, pastilles, capsules and the like may comprise any of the following constituents or similar compounds: a filler such as microcrystalline cellulose (MCC) or mannitol; a binding agent such as hydroxypropyl cellulose (HPC), tragacanth gum or gelatin; an excipient such as starch or lactose; a disintegrating agent such as alginate or corn starch; a lubricant such as magnesium stearate, a sweetener such as sucrose or saccharose; or a flavoring such as peppermint or methyl salicylic acid.

When a dosage form in the form of a capsule is used, it may comprise, in addition to the above constituents, a liquid carrier such as oil. Further, the dosage forms may be designed with, for instance, coating layers from sugar, shellac or other agents. The constituents of the pharmaceutical composition are preferably chosen such that they do not reduce the desired activity of the sphingomyelin and/or lysosphingomyelin.

According to embodiments of the present invention, a sphingomyelin and/or lysosphingomyelin which is isolated from a natural source or synthetically manufactured or a fraction from a suitable source, which is enriched with sphingomyelin and/or lysosphingomyelin, may be used in all types of foods, food products, nutritional supplements and medicaments or pharmaceutical compositions. Preferably, a sphingomyelin and/or lysosphingomyelin is used in dairy products, including, for instance, custard, yogurt, cheese, spreads, drinks, desserts. Diet products can also suitably be enriched with a sphingomyelin and/or lysosphingomyelin.

A sphingomyelin and/or lysosphingomyelin or the pharmaceutically suitable salt thereof can also be administered in the form of, for instance, an elixir, a suspension, a syrup, a dairy product such as a butter or a yogurt, a wafer or a chewing gum.

In a pharmaceutical preparation according to the invention, a sphingomyelin and/or lysosphingomyelin, or a precursor, a derivative or a pharmaceutically suitable salt thereof is used in an amount of 0.001 to 99.9 wt. %, preferably of 0.01 to 10 wt. %, and more preferably of 0.1 to 1 wt. %.

A pharmaceutical preparation according to the invention can also be used to improve the uptake of nutrients which are already naturally found in food, or which are additionally ingested. For instance, a pharmaceutical preparation according to the invention can be used to improve the uptake of pharmaceutically active substances which are ingested as separate preparations. Preferably, a sphingomyelin and/or lysosphingomyelin, or a precursor, a derivative or a pharmaceutically suitable salt thereof is used in a pharmaceutical preparation to improve the uptake of the pharmaceutically active substance present in that same preparation.

A sphingomyelin and/or lysosphingomyelin, or a precursor, a derivative or a pharmaceutically suitable salt thereof can further be used in a food or nutritional supplement. A nutritional supplement is defined as a composition which can be consumed supplementary to the normal food and which comprises constituents which are not, or to an insufficient extent, found in the normal food and of which sufficient or increased consumption is desired. However, the composition of a food does not essentially differ from a nutritional supplement.

A food or nutritional supplement according to the invention contains a content of a sphingomyelin and/or a lysosphingomyelin higher than would naturally or normally or without human intervention be present in such a food or nutritional supplement or would be found therein. This increased content of a sphingomyelin and/or a lysosphingomyelin can be the result of the specific addition of a sphingomyelin and/or lysosphingomyelin to a food composition which does not normally comprise this sphingomyelin and/or lysosphingomyelin, i.e. by an enrichment of this food with a sphingomyelin and/or lysosphingomyelin. A food enriched with sphingomyelin and/or lysosphingomyelin can also be created by the specific addition of a sphingomyelin and/or lysosphingomyelin to a food which normally already comprises sphingomyelin and/or lysosphingomyelin, but whose concentration or content is increased, by the addition, to values which are not normally present in such a composition.

Because the contents of sphingomyelin and lysosphingomyelin greatly differ in different foods, there is no one general value for the content which will involve an increased content of a sphingomyelin and/or lysosphingomyelin or a food enriched therewith. In, for instance, milk, which normally contains quite a lot of sphingomyelin, an increased or enriched content will relate to a higher content than in, for instance, a potato in which hardly any sphingomyelin is present.

A food or nutritional supplement according to the invention enriched with sphingomyelin and/or lysosphingomyelin may very suitably comprise 0.001 to 99.9% of a sphingomyelin and/or lysosphingomyelin. In a preferred embodiment, such a food or nutritional supplement comprises 0.05 to 50 wt. %, preferably 0.1 to 10 wt. %, more preferably 1 to 5 wt. % of a sphingomyelin and/or lysosphingomyelin or derivatives, precursors or suitable salts thereof.

In order to make a food or nutritional supplement comprising a sphingomyelin and/or lysosphingomyelin suitable for consumption, constituents which, for instance, improve the texture, taste or smell may be added to it. A skilled person is familiar with the different sources of protein, carbohydrate and fat which may be used in nutritional supplements and foods according to the invention and with the possible sweeteners, vitamins, minerals, electrolytes, colorings, aromatic substances, flavorings, spices, fillers, emulsifiers, stabilizers, preservatives, antioxidants, dietary fibers and other constituents for foods which can be added to improve the taste or the texture of a food. The choice for such constituents is a matter of formulation, design and preference. The amounts of such constituents which can be added are known to a skilled person, while the choice may, for instance, be guided by the recommended daily amounts (RDA doses) for children and adults.

Doses for ingestion of the food or nutritional supplement may vary in size and are not limited to the values corresponding to the recommended amounts. Herein, the term “nutritional supplement” is not intended to be limited to a specific weight or specific dose.

A composition of a food or nutritional supplement according to the invention may, essentially, have any form suitable for consumption by humans or animals. In one embodiment, the composition has the form of a dry powder which can be suspended, dispersed or emulsified in an aqueous liquid such as water, coffee, tea, broth or fruit juice. For this purpose, such a powder may be provided in a unit dose package.

In an alternative preferred embodiment, the composition can be tabletted in the form of a dry powder. For this purpose, a composition for a nutritional supplement according to the invention may very suitably be provided with fillers, such as microcrystalline cellulose (MCC) and mannitol, binding agent, such as hydroxypropyl cellulose (HPC), and lubricants, such as stearic acid or other excipients.

A composition of a food or nutritional supplement according to the invention may also be provided as a liquid food preparation in which the solid constituents are suspended, dispersed or emulsified in an aqueous liquid. Such a composition may be mixed directly into a food or may, for instance, be extruded and be processed to granules or other forms.

In an alternative embodiment, a food or nutritional supplement may be designed in the form of a solid food, such as a bar, cookie or a roll.

If a food according to the invention is used as animal feed, the food may, for instance, be prepared in the form of a powder, a granule, a wafer, a mash, a lump, a pulp, a paste, a flake, a cake, a (lick) block, a suspension or a syrup.

For administration to humans, a sphingomyelin and/or lysosphingomyelin can very suitably be prepared in the form of a nutritional supplement.

The present invention further relates to a method for preparing a pharmaceutical preparation to improve the uptake of a nutrient or pharmaceutically active substance, comprising incorporating a sphingomyelin and/or lysosphingomyelin, or a precursor, a derivative or a pharmaceutically suitable salt thereof as active substance in a pharmaceutical composition.

The preparation of a pharmaceutical composition may very suitably take place by mixing all separate constituents such as fillers, binding agents, lubricants and, optionally, other excipients together with a sphingomyelin and/or lysosphingomyelin or a precursor, a derivative or a pharmaceutically suitable salt thereof, and processing the mixture obtained to a pharmaceutical preparation.

The present invention further relates to a method for preparing a food or nutritional supplement according to the invention, comprising enriching a food or nutritional supplement with a sphingomyelin and/or lysosphingomyelin or a precursor, a derivative or a pharmaceutically suitable salt thereof

In one embodiment, the invention provides a method for preparing a food or nutritional supplement enriched with a sphingomyelin and/or lysosphingomyelin, comprising incorporating a sphingomyelin and/or lysosphingomyelin or a precursor, a derivative or a pharmaceutically suitable salt thereof in a food or nutritional supplement, preferably to a content of 0.01 to 99.9 wt. %, more preferably to a content of 0.05 to 50 wt. %, still more preferably to a content of 0.1 to 10 wt. %, and most preferably to a content of 1 to 5 wt. %. The amount of sphingomyelin and/or lysosphingomyelin incorporated in a food according to the invention depends on both the compound used itself and the use, and a skilled person will be able to determine this amount in the context of the present description.

In a method for preparing a food according to the invention, the food can first be prepared separately and then be combined with a sphingomyelin and/or lysosphingomyelin in order to obtain a food according to the invention, with the sphingomyelin and/or lysosphingomyelin being incorporated into the food. The food can be separately prepared in advance in known manners such as mixing, frying, deep-frying, cooking, steaming, roasting or poaching and can be cooled down, if necessary, before combining it with a lipid in order to obtain a food according to the invention. According to another usable embodiment, during the preparation of the food, a sphingomyelin and/or lysosphingomyelin is incorporated therein as a constituent.

A sphingomyelin and/or lysosphingomyelin, or a precursor, a derivative or a pharmaceutically suitable salt thereof, and also pharmaceutical preparations and foods prepared on the basis thereof are, as said, used to improve the uptake of a nutrient or a pharmaceutically active substance by the intestinal epithelium, preferably the intestinal epithelium of birds, mammals and humans. Herein, improving the uptake is intended to mean that the uptake of this nutrient or this pharmaceutically active substance from the gastrointestinal tract is increased, preferably in the stomach and the small intestine, and still more preferably in the small intestine, because, there, the uptake of small nutrients takes place.

Thus, the present invention also relates to the use of a sphingomyelin and/or lysosphingomyelin, or a precursor, a derivative or a pharmaceutically suitable salt thereof to improve the uptake of a nutrient or pharmaceutically active substance from the gastrointestinal tract. Such a use is, essentially, of a non-medical nature.

Nutrients and pharmaceutically active substances whose uptake is increased under the influence of sphingomyelin and/or lysosphingomyelin are preferably small polar nutrients and pharmaceutically active substances. In the present description, small polar nutrients are understood to mean all mono, di, tri, tetra and pentasaccharides and also amino acids and small peptides with a MW of approximately 50 to approximately 1500 Da, polar vitamins, metal ions, minerals, hormones and all conceivable polar pharmaceutically active compounds. Very suitably, the uptake of bisphosphonates, mannitol and dipeptides is increased by use of a sphingomyelin and/or lysosphingomyelin.

In particular, there is a preference for the use of lysosphingomyelin to improve the uptake of bisphosphonates. There is a particular preference for increasing the uptake of the bisphosphonates dimethyl-ADP and EB1053.

The improvement of the uptake of nutrients is also provided in uses according to the present invention. Here, the increase of the uptake of nutrients can be advantageous, in particular if it is desired that the body weight is increased or when the body needs to take up nutrients more efficiently, for instance in case of undernourishment or convalescence before or after an operation, or in case of exhaustion. Further, the present invention can be used in the fatting of, for instance, poultry, cattle or pigs, where it is also desired that the body weight is increased.

Finally, the present invention relates to the use of a sphingomyelin and/or lysosphingomyehn or a precursor, a derivative or a pharmaceutically suitable salt thereof for preparing a medicine to improve the uptake of a nutrient or pharmaceutically active substance.

The invention will now be illustrated in and by the following examples, which should not be taken as being limitative.

### EXAMPLES

Experimental Procedure

The following sphingolipids were used in the experiments: phytosphingosine, sphinganine, lysosphingomyelin, egg sphingomyelin, sphingosine, cerebroside and ceramide III. As reference substances, D-mannitol (Sigma-Aldrich, St. Louis, USA), caffeine (Sigma-Aldrich) and cholesterol (Sigma-Aldrich) were used.

As radioactive reference substances, [³H]-mannitol (NEN Life Science Products, Boston, USA; specific activity 973 GBq/mmol), [¹⁴C]-caffeine (NEN Life Science Products; specific activity 1894.4 MBq/mmol), [¹⁴C]-testosterone (NEN Life Science Products; specific activity 1983 MBq/mmol), [¹⁴H]-cholesterol (NEN Life Science Products; specific activity 296 GBq/mmol) and [¹⁴C]GlySar (Cambridge Research Biochemicals; specific activity 2098 MBq/mmol) were used.

Preparation of the Solutions

GlySar, mannitol, testosterone, caffeine and cholesterol were tested at a concentration of 10 μM. This concentration was reached by mixing “cold” and radioactively labeled test substrate (mannitol, testosterone and cholesterol) or by testing the radioactively labeled substrate alone (GlySar and caffeine).

The effect on transport and the transport of lipids was tested with 0.02 wt. % in transport medium (except for lysosphingomyelin and cerebroside, of which 0.008 wt. % and 0.01 wt. %, respectively, were used). Stock solutions of the test compounds were prepared in ethanol. The stock solutions were diluted in transport medium to a final concentration of 2% (v/v) ethanol in transport medium.

In general, the experiments were carried out in the presence and in the absence of 0.03 wt. % of bile. If bile was used in an experiment, this is explicitly stated in the results.

Culture of Caco-2 Cells

In the experiments, Caco-2 cells (ATCC, code HTB 37, human large intestine adenocarcinoma) were used which were stored as frozen stock cultures in liquid nitrogen. Subcultures were prepared from these stock cultures for experimental use.

Caco-2 cells are cells from a human intestinal cancer line (human large intestine adenocarcinoma) which can develop into a dense mono cell layer on a surface, such as a filter. This confluent monolayer is generally accepted and used as a model system for the small intestinal epithelium. The cells even develop the microvilli so characteristic of the small intestine.

The Caco-2 cells were grown in culture medium consisting of Dulbecco's modified Eagle medium (DMEM), supplemented by heat-inactivated fetal calf serum (10% v/v), non-essential amino acids (1% v/v), L-glutamine (2 mM) and gentamicin (50 μg/ml). The Caco-2 cells were grown by seeding approximately 2 million cells per 10 ml of culture medium in 75 cm²-tissue culture bottles. Virtually confluent Caco-2 cell cultures (80-90% confluent) were harvested by means of trypsination and resuspended 1 to 10 in fresh culture medium. The cells were routinely grown in a humidified incubator at 37° C. in an atmosphere of air containing 5% of CO₂.

Two-Compartment Transport System

The cultivation of Caco-2 cells in a two-compartment transport system is regularly used to investigate intestinal, epithelial permeability. In this system, the Caco-2 cells differentiate into polarized columnar cells after reaching confluence. The Caco-2 system has been shown to simulate the passive and the active transcellular transport of electrolytes, sugars, amino acids and lipophilic compounds in the small intestine (Hillgren et al., Medical Research Reviews 15:83-109, 1995; Dulfer et al., J. Lipid Res. 37: 950-961, 1996). Also, a clear correlation between the in vivo absorption and the permeability over the monolayer of Caco-2 cells has been reported (Artursson and Karlsson, Biochem. Biophys. Res.Commun. 175:880-885, 1991).

For the present transport studies, Caco-2 cells (passage 34) were seeded on semi-permeable filter inserts (12-well Transwell™ plates, 0.4 μm pores, Corning Costar) at 100,000 cells.per filter (growth surface 1.13 cm² containing 2.5 ml of culture medium). The number of cells was counted using a Bürker-Türk count chamber. The cells on the inserts were grown for 21 days at 37° C. in a humidified incubator in an atmosphere of air containing 5% of CO₂. During such a culture period, the cells undergo an enterocyte-like differentiation.

Measurement of Transepithelial Electrical Resistance (TEER)

After 21 days of cell culture, the transepithelial electrical resistance (TEER) of the monolayers was used to check on the growth and differentiation of the Caco-2 cells on the filters (Duizer et al., J. Contr. Release 49: 39-49, 1997). The TEER is a measure for the differentiation status of the monolayer and was measured by means of the Millicell-ERS epithelial volt ohm meter (Millipore Co., Bedford, USA). Only monolayers having a TEER higher than 500 Ω.cm⁻² were used in the transport experiments.

Cytotoxicity Assay

Before use, the Caco-2 cells were obtained from a subculture and seeded in sterile 24-well cell culture cluster plates at approx. 100,000 cells per well (growth surface per well is approx. 2 cm²); 1 ml of cell suspension per well. After seeding, the cell cultures were grown for at least 24 hours at 37° C. in a humidified incubator with 5% of CO₂ in air.

Prior to the exposure of the Caco-2 cells to dilutions of the test substrates, the culture medium was removed from the wells. Amounts of 1 ml of each test dilution were transferred to wells with virtually confluent Caco-2 cells (in duplo per test). After exposure, the test dilution was extracted and the cytotoxicity of the test solution was evaluated for the cultures by MTT conversion.

The MTT assay (Mosmann, T. 1983. J. Immunol. Methods 65:55-63) was used to determine the cytotoxicity after the exposure. This assay determines the viability of the cells by determining their metabolic possibilities to reduce MTT in the corresponding MTT-formazan product. In short, the cells were incubated for 1 hour in 1 ml of culture medium with 0.5 mg of MTT/ml. After this incubation period, the MTT medium was carefully removed. The MTT-formazan product, formed by the living cells, was extracted during at least 1 hour using 1 ml of DMSO. The absorption was measured at a wavelength of 540 nm and a reference wavelength of 655 nm by using a Biorad multi-well plate reader.

Three filter inserts were used per experimental group. The integrity of all monolayers of Caco-2 cells used in this study was determined by measuring the transepithelial electrical resistance (TEER).

The culture medium was removed from the filter inserts (apical compartment) and from under the filter inserts (basolateral compartment). The inserts were washed using phosphate-buffered saline (PBS) and were moved to a new 12-well plate, whose wells had been filled with 1.8 ml of transport medium (DMEM without phenol red, non-essential amino acids (1% v/v), L-glutamine (2 mM) and gentamicin (50 μg/ml)). The transport study was started by filling the apical chamber with 600 μl of the dosed solution. A sample of 100 μl from the apical compartment was collected directly after administration of the dosage. The 12-well plate with the filter inserts was incubated on a rotating platform (60 rpm) in a humidified incubator at 37° C. under an atmosphere of 5% of CO₂ in air. Samples (500 μl) from the basolateral compartment were collected at 15, 30, 60 and 120 min after the start of the transport study. Directly after sampling, the original volume in the receptor compartment was brought back to the original value by adding fresh transport medium. After 120 min, a sample of 100 μl was taken from the apical chamber.

Analysis

The radioactivity in all samples was determined as DPM (disintegrations per minute), using an LKB/Wallac S1409 scintillation counter. The amount of radioactivity was determined in the 100-μl samples of the dose compartments and in the 500-μl samples of the receptor compartments.

TABLE 1
Transport of mannitol, GlySar, caffeine, testosterone, cholesterol and the bisphosphonates
dimethyl-ADP (d-ADP) and EB-1053 through confluent Caco-2 cell layers.

| | | Nutrient (10 | Transport velocity | Velocity measured/ |
|---|---|---|---|---|
| Test compound | Additive | μM) | (cm · hour⁻¹ × 10⁻⁷) | Control velocity |
| phytosphingosine | | mannitol | 21.9 ± 1.8 | 1 |
| | | GlySar | 41.0 ± 8.4 | 1 |
| | | caffeine | 533 ± 27 | 1 |
| | | testosterone | 537 ± 32 | 1 |
| | 0.03% bile | cholesterol | 2.1 (n = 1) | 1 |
| ceramide III | | mannitol | 26.2 ± 7.2 | 1 |
| | | GlySar | 34.5 ± 4.9 | 0.7 |
| | | caffeine | 490 ± 48 | 1 |
| | | testosterone | 503 ± 10 | 1 |
| | | cholesterol | nd | nd |
| lysosphingomyelin | | mannitol | 120 ± 9 | 2.7 |
| | | GlySar | 79.7 ± 28.1 | 1.6 |
| | | caffeine | 559 ± 38 | 1 |
| | | testosterone | 536 ± 21 | 1 |
| | 0.03% bile | cholesterol | 1.25 (n = 1) | 1 |
| | | d-ADP | 922 ± 10 | 4.9 |
| | | d-ADP (10× | 498 ± 8 | 6.1 |
| | | conc.) | | |
| | | EB1053 | 371 ± 17 | 12.2 |
| | | EB1053 (10× | 414 ± 13 | 12.1 |
| | | conc.) | | |
| | 0.03% bile | mannitol | 12.3 ± 1.8 | 1 |
| | 0.03% bile | GlySar | 35.4 ± 2.4 | 1 |
| | 0.03% bile | caffeine | 560 ± 29 | 1 |
| | 0.03% bile | testosterone | 576 ± 24 | 1 |
| sphinganine | 0.03% bile | cholesterol | 1.78 ± 0.53 | 1 |
| sphingosine | 0.03% bile | cholesterol | 2.23 ± 1.15 | 1 |
| egg sphingomyelin | 0.03% bile | cholesterol | 5.84 ± 0.31 | 5.3 |
| glucosylceramide III | 0.03% bile | cholesterol | 0.77 ± 0.27 | 1 |

In the experiments whose results are shown in Table 1, phytosphingosine was added as 0.02 wt. % (0.6 μM), ceramide III as 0.02 wt. % (0.3 μM), lysosphingomyelin as 0.008 wt. % (0.2 μM), sphinganine as 0.02 wt. % (0.6 μM) sphingosine as 0.02 wt. % (0.6 μM), egg sphingomyelin as 0.02 wt. % (0.3 μM) and glucosylceramide III as 0.02 wt. % (0.3 μM).

The increase of the uptake of nutrients and medicaments as a result of the presence of sphingomyelin and/or lysosphingomyelin could be determined by tests with Caco-2 cell lines. The Caco-2 cell layer selectively transports substances such as salts, sugars, amino acids and lipids from the apical to the basolateral side. The uptake of substances such as mannitol, the dipeptide derivative glycylsarosine (GlySar), caffeine, testosterone, cholesterol and bisphosphonate was investigated. Mannitol is used as a marker in Caco-2 transport studies to measure the paracellular transport. Caffeine is used as a marker to measure the transcellular transport. For a number of substances, the tests are described in the examples below.

The experiments show that lysosphingomyelin and sphingomyelin affect the transport velocities. Mannitol is transported approx. 3 times better and GlySar is transported approx. 1.5 times better in the presence of 0.008% (w/v) lysosphingomyelin (sphingosine-phosphorylcholine or sphingosyl-phosphorylcholine [SPC]) on the basis of the Papp values measured. At this same lysosphingomyelin concentration, dimethyl-ADP is transported 5 to 6 times faster and EB-1053 12 times faster. This probably involves paracellular transport because the mannitol transport also increases under the influence of lysosphingomyelin while the caffeine transport is not influenced. Also, the resistance between the apical and basolateral liquids was found to decrease under the influence of lysosphingomyelin.

The tests further showed that, when 19 μg/ml of lysosphingomyelin or 16.6 μg/ml of phytosphingosine was added to the apical compartment, after 120 minutes, 9.8 μg/ml of lysosphingomyelin and 12.9 μg/ml of phytosphingosine could still be demonstrated in the apical liquid, while no lipids could be demonstrated in the basolateral liquids. Thus, no measurable transport of lipids takes place from the apical to the basolateral side. Possibly, the Caco-2 cells take up the lipids.

1. A pharmaceutical preparation to improve the uptake of a nutrient or pharmaceutically active substance from the gastrointestinal tract, comprising sphingomyelin and/or lysosphingomyelin, or a precursor, a derivative or a pharmaceutically suitable salt thereof and one or more excipients.

2. A preparation according to claim 1, wherein the sphingomyelin and/or lysosphingomyelin is lysosphingomyelin.

3. A preparation according to claim 1, wherein the sphingomyelin and/or lysosphingomyelin, or a precursor, a derivative or a pharmaceutically suitable salt thereof is present in an amount of 0.1 to 1 wt. %.

4. A food or nutritional supplement enriched with a sphingomyelin and/or lysosphingomyelin, or a precursor, a derivative or a pharmaceutically suitable salt thereof.

5. A food or nutritional supplement according to claim 4, comprising a content of 0.05-50 wt. % of said sphingomyelin and/or lysosphingomyelin.

6. A food or nutritional supplement according to claim 4, wherein said sphingomyelin and/or lysosphingomyelin is lysosphingomyelin.

7. A method for preparing a preparation according to any one of claims 1-3 and 14, comprising incorporating said sphingomyelin and/or lysosphingomyelin, or a precursor, a derivative or a pharmaceutically suitable salt thereof in a pharmaceutical preparation.

8. A method for preparing a food or nutritional supplement according to any one of claims 4-6, 14 and 15, comprising incorporating said sphingomyelin and/or lysosphingomyelin, or a precursor, a derivative or a pharmaceutically suitable salt thereof in a food or nutritional supplement.

9. A method according to claim 8, wherein said sphingomyelin and/or lysosphingomyelin, or a precursor, a derivative or a pharmaceutically suitable salt thereof is incorporated in said food or nutritional supplement up to a content of 0.01 to 99.9 wt. %.

10. Use of a sphingomyelin and/or lysosphingomyelin, or a precursor, a derivative or a pharmaceutically suitable salt thereof to improve the uptake of a nutrient or pharmaceutically active substance from the gastrointestinal tract.

11. Use according to claim 10, wherein said sphingomyelin and/or lysosphingomyelin is lysosphingomyelin and/or analogs of lysosphingomyelin in which choline is substituted by ethanolamine, serine or inositol.

12. Use according to any one of claims 10-11, wherein said pharmaceutically active substance is bisphosphonate.

13. Use of a sphingomyelin and/or lysosphingomyelin, or a precursor, a derivative or a pharmaceutically suitable salt thereof for preparing a medicine to improve the uptake of a nutrient or pharmaceutically active substance from the gastrointestinal tract.

14. A preparation according to claim 2, wherein the sphingomyelin and/or lysosphingomyelin, or a precursor, a derivative or a pharmaceutically suitable salt thereof is present in an amount of 0.1 to 1 wt. %.

15. A food or nutritional supplement according to claim 5, wherein said sphingomyelin and/or lysosphingomyelin is lysosphingomyelin.
